(19) United States
(12) Patent Application Publication
(10) Pub. No.: US 2023/0237380 A1
(43) Pub. Date: Jul. 27, 2023

(54) MULTIVARIATE OUTLIER DETECTION FOR DATA PRIVACY PROTECTION

(71) Applicant: Siemens Healthcare GmbH, Erlangen (DE)

(72) Inventors: Asmir VODENCAREVIC, Fuerth (DE); Michael Adling, Nuernberg (DE)

(73) Assignee: Siemens Healthcare GmbH, Erlangen (DE)

(21) Appl. No.: 18/159,197

(22) Filed: Jan. 25, 2023

(30) Foreign Application Priority Data
Jan. 27, 2022 (DE) 10 2022 200 919.3

(51) Int. Cl. G06N 20/00; G16H 50/70

(52) U.S. Cl. CPC G06N 20/00; G16H 50/70

(57) ABSTRACT

A computer-implemented data protection method comprising: receiving an input dataset, the input dataset including a plurality of datapoints, at least some of the plurality of datapoints including information usable in combination to identify a patient; performing multivariate outlier detection on the input dataset, the performing including computing anomaly scores for at least a portion of the plurality of datapoints using a multivariate outlier detection algorithm; and identifying, based on the anomaly scores, at least one set of multivariate outliers of datapoints usable in combination to identify the patient.

### CROSS-REFERENCE TO RELATED APPLICATION(S)

The present application claims priority under 35 U.S.C. § 119 to German Patent Application No. 10 2022 200 919.3, filed Jan. 27, 2022, the entire contents of which are incorporated herein by reference.

### FIELD

One or more embodiments of the present invention relate generally to the field of data protection, and more specifically to techniques for multivariate outlier detection for data privacy protection, such as anonymization and pseudonymization.

### BACKGROUND

In today's ubiquitous networked computing systems, there is often a need for granting data access or initiating a data transfer between the parties involved. This is not only the case in data collaborations in general, but especially in clinical collaborations. In one typical scenario, a healthcare provider that serves as a data controller needs to transfer patient data to an industry partner who uses the data for research and/or product development.

Due to its sensitive and personal nature, the privacy of individual patient data must be sufficiently protected, not least because of contractual agreements and/or applicable regulations. One widely applied way to achieve data protection is via data anonymization. In Recital 26 of the General Data Protection Regulation (GDPR), anonymized data is established as “personal data rendered anonymous in such a way that the data subject is not or no longer identifiable”.

The application of this general definition to concrete medical data requires the implementation of measures which go well beyond the stripping of direct patient identifiers, such as patient name, postal code, or phone number. Unusual values in the data should be treated with care, as they are associated with an increased potential of patient re-identification.

In the field of data anonymization, US 2014/0283097 A1 discloses a mechanism for relational context sensitive anonymization of data. A request for data is received that specifies a relational context corresponding to a selected group of persons. The relational context specifies attributes that establish a relationship between the selected persons and distinguish them from persons that are not in the selected group. For the relational context, key attributes in the personal information data are determined and a rarity value for each key attribute is determined. Selected key attributes are then anonymized based on the determined rarity value.

The document “Isolation forest” (Liu F T et al., 8th IEEE international conference on data mining, 2008) discloses An outlier and anomality detection algorithm called Isolation Forest which explicitly isolates anomalies to exploit sub-sampling with linear time complexity and low memory requirement.

Further examples of outlier detection approaches, including elliptic envelope, local outlier factor (LOF) and Shapley additive explanations (SHAP), are disclosed in the documents “A fast algorithm for the minimum covariance determinant estimator” (Rousseeuw P J, Technometrics 1999; 41(3): 212-23), “LOF: identifying density-based local outliers” (Breunig M M et al., In Proc. ACM SIGMOD 2000) and “A Unified Approach to Interpreting Model Predictions” (Lundberg S M et al., Advances in Neural Information Processing Systems 2017; 30: 4765-74).

The document “AppScalpel: Combining static analysis and outlier detection to identify and prune undesirable usage of sensitive data in Android applications” (Meng Z et al., Neurocomputing, Volume 341, 2019, Pages 10-25, ISSN 0925-2312) discloses a privacy-preserving system designed to combine static analysis and outlier detection algorithms to identify and prune undesirable usage of sensitive data in Android apps. AppScalpel estimates the similarity between behaviors of the testing app and behaviors extracted from the popular market apps from the same category. One class support vector machine outlier detection algorithm is employed to identify unusual behavior of the testing app which could jeopardize sensitive or private user data.

Generally speaking, anonymization measures are dependent on the concrete data at hand and should ensure data anonymity in both an univariate and a multivariate sense. The former is concerned with detecting and anonymizing unusual values or outliers in a single variable (e.g. patient age), while the latter addresses data points that could be considered unusual (only) when two or more variables are combined (e.g. body mass index and age).

One example of a univariate outlier in this context might be the patient age of 90 in a dataset where the next highest age is 60. Such unusual values in a single variable are relatively easy to detect by simple data exploration techniques and visualizations (e.g., box plots, histograms, etc.) and measures of variability, such as interquartile ranges.

On the other hand, unusual multivariate outliers can be subtle and much more difficult to detect by conventional methods. For example, a patient dataset with not unusual body-mass index, age and depression score might turn out to be a multivariate outlier in a three-dimensional space defined by these variables. The problem is even more striking in high-dimensional spaces, where many variables not only make it difficult to spot the unusual data points but also hinder an explanation why a data point is anomalous.

### SUMMARY

Against this background, the inventors have identified a technical problem underlying certain embodiments of the present invention, which is to provide improved techniques for detecting multivariate outliers which could compromise data anonymization. Another, related problem addressed by embodiments of the present invention is the lack of insight and/or explanation why a multidimensional data point could be an outlier.

This problem is, in one embodiment, solved by a computer-implemented data protection method, in particular for data anonymization and/or data pseudonymization. The method may comprise receiving an input dataset, wherein the input dataset may comprise a plurality of datapoints. At least some of the datapoints may comprise information that is usable in combination to identify a person, such as a patient. The method may comprise performing multivariate outlier detection on the input dataset, comprising computing anomaly scores for at least a portion of, or all of, the plurality of datapoints using a multivariate outlier detection algorithm. The method may also comprise displaying a ranking of the at least a portion of the datapoints based on the anomaly scores.

Accordingly, as an example, the method may be applied to a dataset comprising multiple data points in a tabular format, wherein rows and columns correspond to patients and their characteristics (variables like age, BMI, laboratory values etc.), respectively. For each row (i.e. patient) and/or in relation to all other rows, the method may compute an anomaly score as described above, which serves as a proxy for this row's uniqueness. Furthermore, by sorting the rows according to their computed anomaly scores, a ranking is established pointing out which rows are the most and which the least specific.

Accordingly, multivariate outlier detection may be used herein to recognize isolated datapoints in a dataset which could pose a threat for data privacy as they could be potentially used for (indirect) re-identification of a person, such as a patient. An anomality score, as used herein, may be any value, e.g., a numerical value, that indicates a degree to which the respective datapoint deviates from the rest of the data points in the dataset.

The multivariate outlier detection algorithm may be a machine learning-based algorithm, which makes the method particularly adaptable to complex input datasets. In addition or alternatively, the multivariate outlier detection algorithm may be selected from the group comprising: isolation forest, elliptic envelope, fast-minimum covariance determinant estimator, and/or local outlier factors. These algorithms are particularly suitable for detecting multivariate outliers.

In one aspect of an embodiment of the present invention, the method may further comprise de-identifying at least one of the datapoints. The de-identifying may comprise one or more of: removing a datapoint, rounding a value of a datapoint, categorizing a datapoint and/or transforming a datapoint.

The de-identifying may be performed in a fully or essentially fully automated fashion. Alternatively, the method may comprise receiving user input for de-identifying at least one of the datapoints, wherein the de-identifying is based on the received user input. This way, the user is enabled with an elaborate set of possible actions to pseudonymize and/or anonymize the data.

In another aspect, performing the multivariate outlier detection may comprise computing anomaly scores for the plurality of datapoints using a plurality of different multivariate outlier detection algorithms, preferably based on a user-selectable preference. This way, the respective advantages of each multivariate outlier detection algorithm may be combined into an even more elaborate analysis.

Displaying the ranking may comprise displaying a ranking for each multivariate outlier detection algorithm. This way, the user can switch between different algorithms to compare their results.

Displaying the ranking may also comprise displaying a ranking based on the union and/or the intersection of the results of the multivariate outlier detection algorithms.

In one scenario, the user may have some knowledge about the data, e.g., she is a domain expert such as a clinician or a clinical researcher. In this case, if:

1a) Data follows a Gaussian distribution, an elliptic envelope may be selected as a preferred method.
1b) Data has low to moderately high dimensionality (=number of variables) relative to the number of available data points, then local outlier factor may be selected, as it may give the best results.
1c) In all other cases, isolation forest might give the most reliable results and may thus be selected. However, it might give comparable or even better results in cases 1b) and 1c) than elliptic envelope and local outlier factor, respectively (the “no free lunch” theorem holds here as well as it all depends on the specific dataset).

In another scenario, the user has no knowledge about the dataset. In this case, he might want to apply all three (and potentially further) algorithms and compare their results. If the user is more conservative, he might want to check the union of the results of all algorithms, in order to “catch” as many specific data points as possible, which should then be removed, transformed etc. Otherwise, an intersection of the results might be selected to gain more confidence that the most significant outliers (as detected by all algorithms) are to be identified treated. In a small data setting this might be a preferred choice especially if identified outliers would need to be removed.

In another aspect, the method may further comprise executing an explainable AI module, such as shapley additive explanations, SHAP. The method may comprise displaying a result produced by the explainable AI module together with the ranking. This way, the user may better understand why a datapoint was associated a higher (or lower) outlier score (e.g. that the combination of values of age, BMI and depression score is unusual).

In yet another aspect of an embodiment of the present invention, the method may further comprise performing univariate outlier detection on the input dataset.

In addition or alternatively, the method may further comprise performing direct identifier detection on the input dataset, preferably using a natural language processing algorithm. This way, directly identifiable single datapoints can be reliably detected, e.g. the patient name. The results may be automatically deleted, anonymized, pseudonymized, flagged to the user, or the like.

Preferably, the multivariate outlier detection algorithm to be used is user-selectable. Preferably, the input dataset is a multi-dimensional or even a high-dimensional dataset.

An embodiment of the present invention also provides a computer-implemented data protection method which may perform and of the above-disclosed methods and in addition generates an output dataset. In the output dataset, those datapoints which comprise information that is usable in combination to identify a person, such as a patient, may be de-identified. Accordingly, this aspect ensures that the output dataset does not comprise any sensitive data. The output dataset may be displayed on an electronic display.

In one aspect, the input dataset may be received from a first device at a second device, the multivariate outlier detection may be performed by the second device, and the output dataset may be transmitted from the second device to the first device. In addition or alternatively, the output dataset may be used for the compilation of an anonymized database. Such a database may be associated with a particular input dataset, or may consolidate information relating to a plurality of input datasets. In this regard, the input datasets with outliers would not be adopted to create the anonymized data base.

Also provided is a data processing apparatus or system comprising a means, device, mechanism and/or apparatus for carrying out any of the methods disclosed herein.

An embodiment of the present invention provides a computer program comprising instructions which, when the program is executed by a computer, cause the computer to carry out any of the methods disclosed herein.

According to an aspect, a computer-implemented data protection method is provided. The method comprises a plurality of steps. One step is directed to receiving an input dataset, wherein the input dataset comprises a plurality of datapoints, at least some of which comprise information that is usable in combination to identify a person, such as a patient. Another step is directed to perform a multivariate outlier detection on the input dataset, comprising computing anomaly scores for at least a portion of the plurality of datapoints using a multivariate outlier detection algorithm. Another step is directed to identify, based on the computed anomaly scores, at least one set of multivariate outliers of datapoints which are usable in combination to identify a person (or, in brief, a set of datapoints which are usable in combination to identify a person).

The input dataset may comprise patient data of one or more patients. In particular, the input dataset may comprise the electronic medical record(s) of one or more patients. The set(s) of multivariate outliers may respectively comprise or be constituted by datapoints belonging to an individual patient. The datapoints of a set of multivariate outliers may be such that they do not constitute outliers if taken alone. Only if taken in conjunction, the datapoints of a set of multivariate outliers my constitute a data privacy risk as they might lead to the identification of an individual person or patient.

The input dataset may be anonymized. This may mean that any personal information useful for directly identifying a person or patient has been removed. Such personal information may relate to the patient's name, the treating physician's name, the treating organization and so forth.

With the set of multivariate outliers, datapoints are automatically identified which are, if taken together, so remarkable that they may lead to the identification of individuals (persons or patients). By automatically identifying such datapoints, the data security may be improved especially if the input dataset is to be shared between different healthcare organizations.

According to an aspect, the method further comprises automatically de-identifying the at least one set of multivariate outliers in the input data set so as to generate a processed dataset, and providing the processed dataset.

In other words, an automatic processing is provided for increasing the data privacy protection in healthcare datasets. The processed dataset may be conceived as a de-identified dataset. In suchlike de-identified dataset, additional data protection measures have been applied besides ordinary anonymization.

According to an aspect, the step of de-identifying comprises one or more of: removing a datapoint, rounding a value of a datapoint, substituting a datapoint, categorizing a datapoint, and/or transforming a datapoint.

With the above actions, it can be ensured that datapoints which might lead to an identification of individuals are deleted or masked without compromising the overall usability of the dataset.

According to an aspect, the method further comprises displaying the input dataset in a user interface with the set of multivariate outliers highlighted.

With the displaying step, a user may be pointed to datapoints which, in combination, could pose a data privacy risk. This brings the user in a position to take appropriate actions such as deleting or altering the set of multivariate outliers.

According to an aspect, the method further comprises receiving a user input from the user via the user interface, the user input being directed to the set of multivariate outliers, processing the input dataset according to the user input so as to generate a processed dataset, and providing the processed dataset.

In other words, the method offers the possibility to de-identify the input dataset in a continued human-machine interaction. For instance, with the user input, a user can verify whether or not a set of multivariate outliers constitutes a data privacy risk and, if so, can decide how the outliers are to be dealt with for providing the de-identified processed dataset.

According to an aspect, in the step of identifying, a set of datapoints is identified as the set of multivariate outliers of datapoints if the anomaly score of the set of datapoints exceeds a predetermined threshold.

The predetermined threshold may be set in advance of the processing. In particular, the predetermined threshold may be a machine-learned value which proved useful to discriminate between sets of datapoint which constitute a data privacy risk and sets of datapoints which do not. In addition or as an alternative, the predetermined threshold may be set or modified by a user in order to tune the sensitivity of the detection of sets of multivariate outliers. In any case, with the threshold, an objective criterium is provided for the identification of sets of multivariate outliers.

According to an aspect, in the step of identifying, a plurality of sets of multivariate outliers of datapoints is identified, and the method further comprises displaying a ranking of the plurality of sets based on the respective anomaly scores in a user interface for a user.

Accordingly, a user is provided with an indication which elements of the input dataset are most pertinent in terms of a possible data privacy breach.

According to an aspect, the method further comprises performing a univariate outlier detection on the input dataset, comprising computing singular anomaly scores for at least a portion of the plurality of datapoints using a univariate outlier detection algorithm. Another step is directed to identify, based on the computed singular anomaly scores, one or more individual datapoints which are usable to identify a person alone.

With that, also individual values may be detected laying beyond the norm, and which might therefore alone lead to an identification of a person or patient. According to some examples, the step of identifying the one or more individual datapoints may precede the steps for identifying the sets of multivariate outliers.

For calculating the singular anomaly scores and identifying the one or more datapoints, in principle the same algorithms may be used as used for identifying the sets of multivariate outliers. However, according to some examples, detecting univariate may simply involve calculating an average for a given parameter such as a patient's age. A singular anomaly score could then be based on a deviation of individual datapoints from the average.

According to an aspect, the multivariate outlier detection algorithm and/or the univariate outlier detection algorithm are machine learning-based algorithms.

According to an aspect, the multivariate outlier detection algorithm and/or the univariate outlier detection algorithm are selected from the group comprising: isolation forest, elliptic envelope, fast-minimum covariance determinant estimator, and/or local outlier factors.

According to an aspect, the method further comprises receiving a user input directed to at least one of the datapoints in the set of multivariate outliers, the user input being optionally directed to de-identify the at least one of the datapoints, and using the received user input for training the multivariate outlier detection algorithm.

With that, a further training of the multivariate outlier detection algorithm can be effected which may further improve the processing.

According to an aspect, performing the multivariate outlier detection comprises computing partial anomaly scores for the plurality of datapoints using a plurality of different multivariate outlier detection algorithms, preferably based on a user-selectable preference, and computing anomaly scores comprises aggregating the partial anomaly scores to generate the anomaly scores.

For instance, aggregating may be based on calculating average anomaly scores based on the partial anomaly scores. According to other examples, anomaly scores may be calculated based on a weighted sum of the partial anomaly scores.

By basing the calculation of anomaly scores on different detection algorithms, the outcome may be improved as systematic shortcomings of individual algorithms for individual parameters may be balanced.

According to an aspect, the plurality of different multivariate outlier detection algorithms used may be selected by a user. With that, a user may individually configure which algorithms are to be used.

According to an aspect, the method further comprises executing an explainable AI module, such as shapley additive explanations, SHAP; and optionally, displaying a result produced by the explainable AI module.

According to as aspect, the method further comprises performing univariate outlier detection on the input dataset. According to as aspect, the method further comprises performing direct identifier detection on the input dataset, preferably using a natural language processing algorithm.

According to as aspect, the input data set comprises at least one electronic medical health record of a patient and, optionally, a plurality of medical health records of a plurality of patients.

According to an aspect, a computer-implemented method for providing an output dataset is provided. The method comprises generating an output dataset, wherein, in the output dataset, those datapoints which comprise information that is usable in combination to identify a person, such as a patient, are de-identified based on the anomaly scores and/or the singular anomaly scores.

According to an aspect, the input dataset is received from a first device at a second device remote from the first device, the multivariate outlier detection and, optionally, the univariate outlier detection, is performed by the second device, and the output dataset is transmitted from the second device to the first device.

According to other examples, the multivariate outlier detection and, optionally, the univariate outlier detection, is performed by the first device prior to transmitting the output dataset to the second device.

According to some examples, the first and second devices are connected via a network such as an intranet or the internet.

According to some examples, the first device may be a client computing device, such as a workstation or laptop or tablet or smartphone of a user, and the second device may be a server device, such as a cloud server. The first device may be located in a first computing environment such as an intranet of a healthcare organization. The second device may be located outside of the first computing environment.

According to an aspect, a data processing apparatus for providing an output dataset comprising an interface unit and a computing unit is provided. The interface unit is configured for receiving an input dataset, the input dataset comprising a plurality of datapoints, at least some of which comprise information that is usable in combination to identify a person, such as a patient. The computing unit is configured to perform a multivariate outlier detection on the input dataset, comprising computing anomaly scores for at least a portion of the plurality of datapoints using a multivariate outlier detection algorithm. The computing unit is further configured to identify, based on the computed anomaly scores, at least one set of multivariate outliers of datapoints which are usable in combination to identify a person. The computing unit is further configured to automatically de-identify the at least one set of multivariate outliers in the input dataset so as to generate a processed dataset. The computing unit is further configured to provide the processed dataset via the interface.

The computing unit may comprise an outlier detection unit configured to host, run and/or apply the outlier detection algorithm. The computing unit may comprise an identification unit configured to identify sets of multivariate outliers of datapoints. The computing unit may further comprise a de-identification unit for de-identifying sets of multivariate outliers in input datasets.

The computing unit may be realized as a data processing system or as a part of a data processing system. Such a data processing system can, for example, comprise a cloud-computing system, a computer network, a computer, a tablet computer, a smartphone and/or the like. The computing unit can comprise hardware and/or software. The hardware can comprise, for example, one or more processors, one or more memories and combinations thereof. The one or more memories may store instructions for carrying out the method steps according to embodiments of the present invention. The hardware can be configurable by the software and/or be operable by the software. Generally, all units, sub-units or modules may at least temporarily be in data exchange with each other, e.g., via a network connection or respective interfaces. Consequently, individual units may be located apart from each other.

The interface unit may comprise an interface for data exchange via internet connection. The interface unit may be further adapted to interface with one or more users of the system, e.g., by displaying the result of the processing by the computing unit to the user (e.g., in a graphical user interface) or by allowing the user to adjust parameters for de-identifying input datasets.

According to another aspect, the present invention is directed to a computer program product comprising program elements which induce a computing unit of a system for identifying a set of multivariate outliers of datapoints in an input dataset to perform the steps according to one or more of the above method aspects, when the program elements are loaded into a memory of the computing unit.

According to another aspect, the present invention is directed to a computer-readable medium on which program elements are stored that are readable and executable by a computing unit of a system identifying a set of multivariate outliers of datapoints in an input dataset according to one or more method aspects, when the program elements are executed by the computing unit.

The realization of one or more embodiments of the present invention by a computer program product and/or a computer-readable medium has the advantage that already existing providing systems can be easily adapted by software updates in order to work as proposed by one or more embodiments of the present invention.

The computer program product can be, for example, a computer program or comprise another element next to the computer program as such. This other element can be hardware, e.g., a memory device, on which the computer program is stored, a hardware key for using the computer program and the like, and/or software, e.g., a documentation or a software key for using the computer program. The computer program product may further comprise development material, a runtime system and/or databases or libraries. The computer program product may be distributed among several computer instances.

### BRIEF DESCRIPTION OF THE DRAWINGS

The disclosure may be better understood by reference to the following drawings, in which

FIG. 1 schematically depicts a conceptual overview of a data anonymization and pseudonymization tool in accordance with embodiments,

FIG. 2 schematically depicts an embodiment of a system for identifying a set of multivariate outliers in an input dataset, and

FIG. 3 schematically depicts a method for identifying a set of multivariate outliers in an input dataset according to an embodiment.

### DETAILED DESCRIPTION

The above mentioned attributes, features, and advantages of this invention and the manner of achieving them, will become more apparent and understandable (clear) with the following description of embodiments of the present invention in conjunction with the corresponding drawings. Embodiments of the present invention provide improved multivariate outlier detection techniques for data privacy protection. Throughout the present disclosure, the term “data anonymization” may be used, which is to be interpreted broadly, e.g., in the sense of removing personally identifiable information from data sets so that the people whom the data describe remain anonymous, but should also cover other, possibly weaker, de-identification procedures such as, without limitation, pseudonymization.

Certain embodiments of the present invention are based on machine learning algorithms for detecting multivariate outliers in multivariate datasets which could pose a threat for data privacy as they could be potentially used for indirect re-identification of patients. In this context, “multivariate” is understood as referring to more than one dimension. This may relate e.g. to variables, e.g. to “columns” in data tables. Datapoints may relate to “rows”, e.g. patients or their follow-ups.

Algorithms suitable for detecting multivariate outliers in embodiments of the present invention include, without limitation, isolation forest, the fast-minimum covariance determinant estimator (elliptic envelope), and/or local outlier factors. Another example is described in “Estimating the support of a high-dimensional distribution” by Schölkopf, Bernhard, et al. (Neural computation 13.7 (2001): 1443-1471). Generally speaking, these algorithms compute an anomaly score for each datapoint based on its deviation from other points (the used distance metric is algorithm-specific, e.g. it can be Cosine, Euclidean, Manhattan, Mahalanobis etc.).

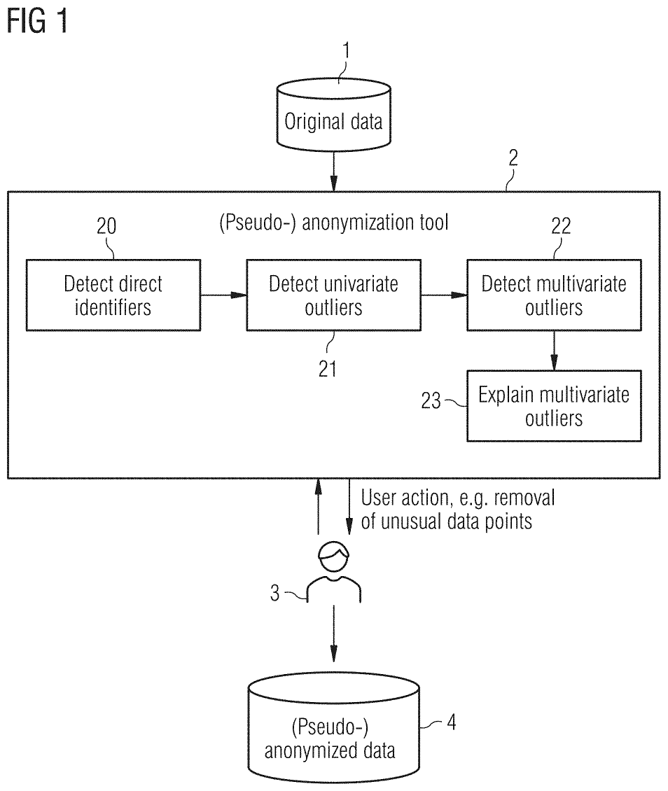

FIG. 1 illustrates a conceptual overview of a data anonymization and pseudonymization tool/system in accordance with an embodiment of the present invention. The system may be part of a larger computing system, which may comprise a memory, a processing unit and a visualization unit (e.g., for showing SHAP AI explanations, ranked data points, etc.). As can be seen, a software tool 2 (labelled “(Pseudo-) anonymization tool” in FIG. 1) is provided which comprises (functional) modules for performing various privacy-protection related tasks. It shall be noted that FIG. 1 depicts an elaborated tool which combines a number of modules. However, embodiments of the present invention are conceivable that include only one or a subset of the depicted modules.

As can be seen in FIG. 1, the tool 2 receives input data 1, i.e. data to be anonymized, as input.

Block 22 represents a module that serves to detect multivariate outliers in the input data 1. To this end, the module may implement one or more of the above-mentioned multivariate outlier detection algorithms. As already mentioned, these algorithms may compute an anomaly score for each datapoint based on its deviation from other datapoints.

Based on computed anomaly scores, a ranking of data points may be established, which shows which datapoints are more sensitive (i.e. specific, unusual) than others. In other words, module 22 may receive data to-be-protected as an input 1 and provide a ranking of datapoints with respect to their multivariate outlier score as an output. In one implementation of the ranking, based on the value of anomaly score, datapoints from the dataset (e.g. “rows”) may be sorted from lowest to highest. The datapoint with the lowest score value may get the rank 1 as the most prominent outlier, the next one the rank 2 etc. In other implementations, there might be algorithm implementations where outliers get higher anomaly scores. For example, in the scikit-learn python library, the lower the score, the more abnormal the data point is.

The user 3 may then remove them or apply other measures, such as rounding, categorization, variable transformation by applying logarithm, square root and other variance-stabilizing transformations, winsorizing, trimming etc. To make them less specific in the final output data 4. One example would be rounding a real number such BMI to one decimal place or an integer value. In another example a numerical variable such as age could be categorized into categories [0, 10), [11, 20), [21, 30) etc.

Optionally, the user may set a parameter controlling the expected proportion of unusual datapoints in the given dataset (e.g. 1% or 5%) and get the corresponding number of datapoints with the highest outlier ranking highlighted in the tool 2.

The user may select to see the multivariate outliers as determined by:
one, several or all single algorithms
the union of results of multiple algorithms; and/or
the intersection of results of multiple algorithms

In addition to multivariate outlier detection algorithms, the software tool 2 may also incorporate an explainable AI module 23, e.g. based on a framework such as shapley additive explanations (SHAP). This enables automated reasoning and helps the user to understand why a data point was associated with a higher (or lower) outlier score (e.g. that the combination of values of age, BMI and depression score is unusual).

The software tool 2 may also incorporate a module 21 with techniques for detecting univariate outliers in single variables, such as interquartile ranges, and/or visualizations, such as box plots and/or histograms. The module 21 may be used independently or in combination with the module 22 for multivariate outlier detection.

Moreover, the tool 2 may also include a module 20 for detecting direct identifiers, such as without limitation patient name, birth date (and/or other absolute dates), postal code, address, social security number, telephone number etc.

An NLP-based (natural language processing) program may automatically find and mark such variables in the given dataset by checking the format of their values against a predefined extensive list of possible formats for these variables.

The user may inspect the marked variables, verify/change the selection and/or undertake an action such as variable removal from the dataset, yielding a pseudonymized dataset.

Unlike in the prior art, the anomaly score in embodiments of the present invention preferably relates to numerical values, rather than to (the counts of) terms. Also, rarity scores known from the prior art are typically associated with single attributes (i.e. variables or terms), while anomaly scores according to certain embodiments of the present invention are associated with data points (i.e. instances or observations).

Furthermore, the computation of rarity scores in the prior art is typically based on a (supervised) linear regression model, while the anomaly scores of embodiments of the present invention are computed by (unsupervised) multivariate outlier detection algorithms.

Also, some prior art approaches detect outliers and extract knowledge from distributed databases without compromising private information. In these approaches, outlier detection algorithms are typically applied to already protected data (e.g. cryptographically or by applying a private random perturbation matrix, i.e. by addition of random noise). In contrast, in embodiments of the present invention, the purpose of outlier detection algorithms is to achieve data privacy protection.

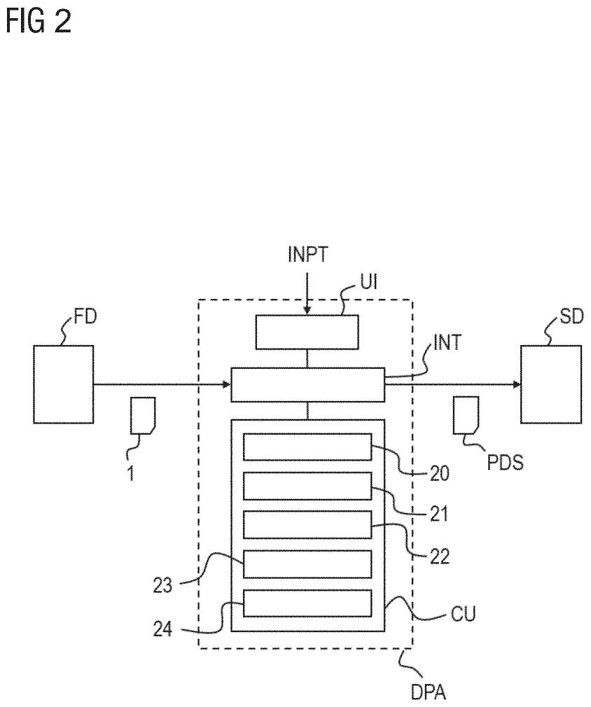

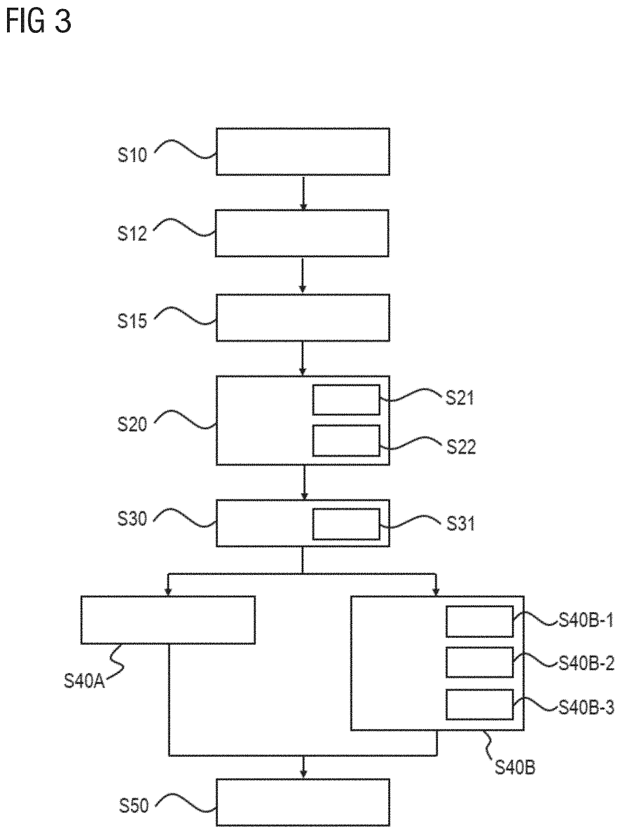

FIG. 2 depicts a data processing apparatus DPA for providing a de-identified or processed dataset PDS based on an input dataset 1. In this regard, the data processing apparatus DPA is adapted to perform the methods according to one or more embodiments, e.g., as further described with reference to FIG. 3.

The data processing apparatus DPA comprises an interface unit INT and a computing unit CU. In particular, the computing unit CU may host and run the software tool 2.

The interface unit INT may be configured for data exchange with other entities or devices. For instance, interface unit INT may be configured to receive the input dataset 1 from one device FD and forward the processed dataset PDS to a second device SD. The interface unit INT may be realized as hardware- or software-interface, e.g., a PCI-bus, USB or fire-wire. Data transfer may be realized using a network connection. The network may be realized as local area network (LAN), e.g., an intranet or a wide area network (WAN). Network connection is preferably wireless, e.g., as wireless LAN (WLAN or Wi-Fi). Further, the network may comprise a combination of different network examples.

According to some examples, the interface unit INT may comprise or be connected to a user interface UI for interfacing with a user of the data processing apparatus DPA. A user of the data processing apparatus DPA, according to some examples, may generally relate to a healthcare professional such as a physician, clinician, technician, and so forth. The user interface UI may comprise a display unit and an input unit via which various user inputs INPT may be received. User interface UI may be embodied by a mobile device such as a smartphone or tablet computer. Further, user interface UI may be embodied as a workstation in the form of a desktop PC or laptop. The input unit may be integrated in the display unit, e.g., in the form of a touch screen. As an alternative or in addition to that, the input unit may comprise a keyboard, a mouse or a digital pen and any combination thereof. The display unit may be configured for displaying the input dataset 1 optionally with any sets of multivariate outliers highlighted and/or a ranking of the multivariate outliers.

The computing unit CU may comprise sub-units 20-24 configured to process the input dataset 1 in order to provide a de-identified output dataset PDS based on a hierarchical de-identification process.

The computing unit CU may be a processor. The processor may be a general processor, central processing unit, control processor, graphics processing unit, digital signal processor, three-dimensional rendering processor, image processor, application specific integrated circuit, field programmable gate array, digital circuit, analog circuit, combinations thereof, or other now known device for processing data. The processor may be a single device or multiple devices operating in serial, parallel, or separately. The processor may be a main processor of a computer, such as a laptop or desktop computer, or may be a processor for handling some tasks in a larger system, such as in a medical information system or server. The processor is configured by instructions, design, hardware, and/or software to perform the steps discussed herein. The computing unit CU may be comprised in the interface unit INT, e.g., in the form of a processor of a tablet, laptop, or workstation computer. Alternatively, computing unit CU may comprise a real or virtual group of computers like a so called ‘cluster’ or ‘cloud’. Such server system may be a central server, e.g., a cloud server, or a local server, e.g., located on a hospital site. Further, computing unit CU may comprise a memory such as a RAM for temporally loading the input dataset 1. According to some examples, such memory may as well be comprised in the interface unit INT.

Sub-unit 20 may be seen as an anonymization module or unit. It is configured to detect direct identifiers of patients in the input dataset 1, such as patient names, birth dates (and/or other absolute dates), postal codes, addresses, social security numbers, telephone numbers etc. To this end, sub-unit 20 may be configured to run an appropriate algorithm or program which is configured to parse the input dataset 1 for direct identifiers. As mentioned, suchlike program may be NLP-based.

Sub-unit 21 may be seen as a univariate outlier detection module or unit. It is configured to detect isolated datapoints in the input dataset 1 which are suited to indirectly identify a patient. This may pertain to isolated values which are such that they identify a person in the input dataset 1 like single outstanding vital parameters or demographic information of a patient. To fulfill this task, sub-unit 21 may be configured to host and run an appropriate detection algorithm which can be, as mentioned, a machine learned algorithm. As such, sub-unit 21 can be conceived as a second stage in the de-identification of the input dataset 1.

Sub-unit 22 may be seen as a multivariate outlier detection module or unit. It is configured to detect sets of a plurality of datapoints in the input dataset 1 which are suited to indirectly identify a patient if taken together. This may pertain to parameter combinations which are suited to pinpoint individual patients in the cohort of the patients represented by the input dataset 1. As explained, sub-unit 22 may be configured to host and run the multivariate outlier detection algorithm according to embodiments herein described. Sub-unit 22 may be conceived as a third stage in the automated de-identification of the input dataset 1 following the initial anonymization and univariate outlier detection.

Sub-unit 23 may be conceived as an explainable AI module or unit. Sub-unit 23 may be configured to host and run algorithms and tools configured to elucidate the basis for the decision making of the involved algorithms in the de-identification process to the user.

Sub-unit 24 may be seen as a de-identifying module or unit. Sub-unit 24 may be configured to de-identify the input dataset 1 based on the findings made by any one of the sub-units 20 to 22. In particular, sub-unit 24 may be configured to delete, erase, substitute, mask, etc. any direct identifiers and indirect identifiers such as univariate and/or multivariate outliers. With that, sub-unit 24 is configured to provide a processed dataset PDS which has been de-identified.

The designation of the distinct sub-units 20-24 is to be construed by way of example and not as a limitation. The sub-units 20-24 may be identical to the modules introduced in connection with software tool 2. Sub-units 20-24 may be integrated to form one single unit (e.g., in the form of “the computing unit”) or can be embodied by computer code segments configured to execute the corresponding method steps running on a processor or the like of the data processing apparatus DPA. Each sub-unit 20-24 and the interface unit INT may be individually connected to other sub-units and/or other components of the data processing apparatus DPA where data exchange is needed to perform the method steps.

The data processing apparatus DPA may be in data exchange with a first device FD and a second device SD. In other words, the data processing apparatus DPA may act as a data filter or gate for data transmissions between the first and second devices FD, SD. Thereby, it can be ensured that protected personal data is not exchanged between first and second devices FD, SD without proper data protection measures.

For instance, the first device FD may be part of a first healthcare organization, computing environment or network. Further, the second device SD may be part of a second healthcare organization, computing environment or network. The first healthcare organization may comprise an internal healthcare network which is not accessible from the outside or only with permission. In this regard, the first device FD may be seen as a gateway to that internal healthcare network of the first organization. Likewise, the second healthcare organization may comprise an internal healthcare network which is not accessible from the outside or only with permission. Here, the second device SD may be seen as a gateway to that internal healthcare network of the second organization.

According to some examples, the first device FD may be part of the healthcare network of a hospital, hospital chain, or private practice etc. Further, the first device FD may be a personal device of a patient, via which a patient can upload personal healthcare data. The second device SD may be part of a central data storage and processing facility configured to aggregate and/or process the healthcare data of a plurality of first organizations and/or a plurality of patients. In particular, the second device SD may be a cloud-based device.

According to some examples, the data processing apparatus DPA may be part of the first device FD or the (internal) healthcare network the first device FD is part of. In other words, the data processing apparatus DPA may be part of the first organization. With that, the data processing apparatus DPA may ensure that protected personal data does not leave the first organization.

According to other examples, the data processing apparatus DPA may be part of the second device SD or the (internal) healthcare network the second device SD is part of. In other words, the data processing apparatus DPA may be part of the second organization. With that, the data processing apparatus DPA may ensure that only data is adopted in the second organization that does not violate data privacy regulations.

FIG. 3 depicts a computer-implemented data protection method according to embodiments of the present invention. The method comprises several steps. The order of the steps does not necessarily correspond to the numbering of the steps but may also vary between different embodiments of the present invention. Further, individual steps or a sequence of steps may be repeated.

At step S10, the input dataset 1 is received. This may involve forwarding the input dataset 1 from the first device FD to the data processing apparatus DPA.

At optional step S12, the input dataset 1 is processed in order to detect data items which are suited to directly identify a patient. Further, such data items may be automatically anonymized at step S12, e.g., by removing or altering these data items. As a result, an anonymized input dataset 1 may be provided at the end of step S12.

At optional step S15, the (optionally: anonymized) input dataset 1 is processed in order to detect univariate outliers in the input dataset 1. That followed, these univariate outliers may be automatically removed or altered at step S15 so as to generate a “pre-de-identified” input-dataset 1 as an intermediate result at the end of step S15.

At step S20 the (optionally: anonymized and/or pre-de-identified) dataset 1 is input in the multivariate outlier detection algorithm in order to provide anomaly scores for combinations of datapoints comprised in the input dataset 1. In this regard, the combinations of datapoints may be limited to combinations of datapoints belonging to one single patient, respectively. Accordingly, an anomaly score of a combination of datapoints may quantify how abnormal this combination of datapoints actually is in the context of the input dataset 1. In other words, an anomaly score may quantify how good an individual may still be indirectly identified based on the underlying combination of datapoints (optionally: despite the anonymization and pre-de-identification measures of steps 12 and 15).

In optional sub-steps S21 and S22, this may be accompanied by running an explainable AI tool in order to give a user an indication why a certain combination of datapoints has a given anomaly score. Specifically, the explainable AI module 23 may be applied so as to provide additive explanations for the detection results provided by the multivariate outlier detection algorithm. At step S22, these additive explanations may be provided to a user, e.g., by displaying them in a user interface comprised in the interface unit INT.

At step S30, sets of multivariate outliers of datapoints are determined based on the anomaly scores. In particular, all combinations of datapoints having anomaly scores larger than a predetermined threshold may be identified as a set of multivariate outliers. Conceptually, these would be those combinations of datapoint which require further processing/de-identification to ensure data privacy. The threshold may be set automatically or by a user of the data processing apparatus DPA. According to some examples, the predetermined threshold may be a learned value which has been learned by the multivariate outlier detection algorithm during training.

At optional sub-step S31 a ranking of pertinent combinations of datapoints may be determined based on the anomaly scores. This ranking may then be displayed to a user via the user interface UI.

The following steps S40A and S40B deal with alternative ways of how a de-identification of the input dataset 1 can be implemented in order to provide the processed dataset PDS. Thereby, the steps S40A and S40B may be applied individually but also in combination.

At step S40A, an automatic de-identification is performed. This may comprise removing at least one datapoint of a set of multivariate outliers, removing an entire set of multivariate outliers, rounding at least one datapoint of a set of multivariate outliers, substituting at least one datapoint of a set of multivariate outliers, categorizing at least one datapoint of a set of multivariate outliers, and/or transforming at least one datapoint of a set of multivariate outliers.

Step S40B deals with a semi-automated de-identification by ways of a continued human-machine interaction. At sub-step S40B-1, the input dataset 1 is displayed in a user interface UI of the interface unit INT with the set(s) of multivariate outliers highlighted. That followed, at sub-step S40B-2 a user input INPT is received from the user via the user interface UI. The user input INPT is directed to at least one set of multivariate outliers. Optionally, the user input INPT may comprise an instruction directed to the de-identification of the at least one set of multivariate outliers such as an indication which de-identification method is to be used. That followed, at sub-step S40B-3, the input dataset 1 is processed according to the user input INPT so as to generate a processed dataset PDS.

Finally, at step S50, the processed dataset PDS is provided. In particular, this may involve forwarding the processed dataset PDS to the second device SD via the interface INT.

The following points are also part of the disclosure:
1. A computer-implemented data protection method, the method comprising:

receiving an input dataset (1), wherein the input dataset (1) comprises a plurality of datapoints, at least some of which comprise information that is usable in combination to identify a person, such as a patient;

performing multivariate outlier detection (22) on the input dataset, comprising computing anomaly scores for at least a portion of the plurality of datapoints using a multivariate outlier detection algorithm; and

displaying a ranking of the at least a portion of the plurality of datapoints based on the anomaly scores.

2. The method of 1, wherein the multivariate outlier detection algorithm is a machine learning-based algorithm.
3. The method of 1 or 2, wherein the multivariate outlier detection algorithm is selected from the group comprising:
isolation forest;
elliptic envelope;
fast-minimum covariance determinant estimator; and/or
local outlier factors.
   4. The method of any of the preceding points, further comprising:

de-identifying at least one of the datapoints;

wherein the de-identifying comprises one or more of:
removing a datapoint;
rounding a value of a datapoint;
categorizing a datapoint; and/or
transforming a datapoint.
   5. The method of 4, further comprising:

receiving user input (3) for de-identifying at least one of the data points, wherein the de-identifying is based on the received user input (3); and

optionally, using the received user input (3) for training the multivariate outlier detection algorithm.

6. The method of any of the preceding points,

wherein performing the multivariate outlier detection (22) comprises computing anomaly scores for the plurality of datapoints using a plurality of different multivariate outlier detection algorithms, preferably based on a user-selectable preference; and

wherein displaying the ranking comprises:
displaying a ranking for each multivariate outlier detection algorithm;
displaying a ranking based on the union of the results of the multivariate outlier detection algorithms; and/or
displaying a ranking based on the intersection of the results of the multivariate outlier detection algorithms.
   7. The method of any of the preceding points, further comprising:

executing an explainable AI module (23), such as shapley additive explanations, SHAP; and

optionally, displaying a result produced by the explainable AI module (23) together with the ranking.

8. The method of any of the preceding points, further comprising:

performing univariate outlier detection (21) on the input dataset.

9. The method of any of the preceding points, further comprising:

performing direct identifier detection (20) on the input dataset, preferably using a natural language processing algorithm.

10. The method of any of the preceding points, wherein the multivariate outlier detection algorithm is user-selectable.
11. The method of any one of the preceding points, wherein the input dataset (1) is a multi-dimensional dataset.
12. A computer-implemented data protection method, the method comprising:

performing the method of any one of 1-11;

generating an output dataset, wherein in the output dataset those datapoints which comprise information that is usable in combination to identify a person, such as a patient, are de-identified.

13. The method of 12, wherein:

the input dataset (1) is received from a first device at a second device;

the multivariate outlier detection (22) is performed by the second device;

and the output dataset is transmitted from the second device to the first device.

14. A data processing apparatus or system configured to carry out the method of claim 1.
15. A computer program comprising instructions which, when the program is executed by a computer, cause the computer to carry out the method of any one of 1-13.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without subdividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Bluray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Nonlimiting examples of the non-transitory computer-readable medium include, but are not limited to, rewriteable nonvolatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable nonvolatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

While the present invention has been illustrated and described in detail with the help of a preferred embodiment, the present invention is not limited to the disclosed examples. Other variations can be deducted by those skilled in the art without leaving the scope of protection of the claimed invention.

What is claimed is:

1. A computer-implemented data protection method, comprising:
receiving an input dataset, the input dataset including a plurality of datapoints, at least some of the plurality of datapoints including information usable in combination to identify a patient;
performing multivariate outlier detection on the input dataset, the performing including computing anomaly scores for at least a portion of the plurality of datapoints using a multivariate outlier detection algorithm; and
identifying, based on the anomaly scores, at least one set of multivariate outliers of datapoints usable in combination to identify the patient.

2. The computer-implemented data protection method according to claim 1, further comprising:
automatically de-identifying the at least one set of multivariate outliers of datapoints to generate a processed dataset; and
providing the processed dataset.

3. The computer-implemented data protection method according to claim 2, wherein the automatically de-identifying includes one or more of:
removing a datapoint;
rounding a value of a datapoint;
substituting a datapoint;
categorizing a datapoint; or
transforming a datapoint.

4. The computer-implemented data protection method according to claim 1, further comprising:
displaying the input dataset on a user interface with the at least one set of multivariate outliers of datapoints highlighted.

5. The computer-implemented data protection method according to claim 4, further comprising:
receiving user input via the user interface, the user input being directed to the at least one set of multivariate outliers of datapoints;
processing the input dataset according to the user input to generate a processed dataset; and
providing the processed dataset.

6. The computer-implemented data protection method according to claim 1, wherein
the identifying includes identifying a set of datapoints as the at least one set of multivariate outliers of datapoints in response to an anomaly score of the set of datapoints exceeding a threshold.

7. The computer-implemented data protection method according to claim 1, wherein
the identifying includes identifying a plurality of sets of multivariate outliers of datapoints; and
the method further includes
displaying a ranking of the plurality of sets of multivariate outliers of datapoints based on respective anomaly scores on a user interface.

8. The computer-implemented data protection method according to claim 1, wherein the multivariate outlier detection algorithm is a machine learning-based algorithm.

9. The computer-implemented data protection method according to claim 1, wherein the multivariate outlier detection algorithm is selected from a group including one or more of:
an isolation forest;
an elliptic envelope;
a fast-minimum covariance determinant estimator; or
local outlier factors.

10. The computer-implemented data protection method according to claim 9, further comprising:
receiving user input directed to at least one datapoint in the at least one set of multivariate outliers of datapoints, the user input being directed to de-identifying the at least one datapoint; and
training the multivariate outlier detection algorithm based on the user input.

11. The computer-implemented data protection method according to claim 1,
wherein the performing of the multivariate outlier detection includes computing partial anomaly scores for at least the portion of the plurality of datapoints using a plurality of different multivariate outlier detection algorithms; and
wherein the computing of the anomaly scores includes aggregating the partial anomaly scores to generate the anomaly scores.

12. The computer-implemented data protection method according to claim 1, further comprising:
executing an explainable AI module; and
displaying a result produced by the explainable AI module.

13. The computer-implemented data protection method according to claim 1, further comprising:
performing univariate outlier detection on the input dataset.

14. The computer-implemented data protection method according to claim 1, further comprising:
performing direct identifier detection on the input dataset.

15. The computer-implemented data protection method according to claim 1, wherein the input dataset includes at least one electronic medical health record of a patient.

16. A computer-implemented data protection method comprising:
performing the computer-implemented data protection method of claim 1; and
generating an output dataset, wherein in the output dataset, datapoints including information that is usable in combination to identify a patient, are de-identified based on the anomaly scores.

17. The computer-implemented data protection method according to claim 16, wherein
the input dataset is received from a first device at a second device, the second device being remote from the first device;
the multivariate outlier detection is performed by the second device; and
the output dataset is transmitted from the second device to the first device.

18. A data processing apparatus for providing an output dataset, the data processing apparatus comprising:
an interface unit configured to receive an input dataset and to provide an output dataset, the input dataset including a plurality of datapoints, at least some of the plurality of datapoints including information that is usable in combination to identify a patient; and
at least one processor configured to
perform a multivariate outlier detection on the input dataset by computing anomaly scores for at least a portion of the plurality of datapoints using a multivariate outlier detection algorithm,
identify, based on the anomaly scores, at least one set of multivariate outliers of datapoints usable in combination to identify the patient, and
automatically de-identify the at least one set of multivariate outliers of datapoints to generate the output dataset.

19. A non-transitory computer program product comprising program elements that, when executed by at least one processor of a system, cause the system to perform the computer-implemented data protection method of claim 1.

20. A non-transitory computer-readable medium storing program elements that, when executed by at least one processor of a system, cause the system to perform the computer-implemented data protection method according to claim 1.

21. The computer-implemented data protection method according to claim 11,
wherein the computing of the partial anomaly scores for at least the portion of the plurality of datapoints is based on a user-selectable preference.

22. The computer-implemented data protection method according to claim 14, wherein the direct identifier detection is performed on the input dataset using a natural language processing algorithm.

23. The computer-implemented data protection method according to claim 15, wherein the input dataset includes a plurality of medical health records of a plurality of patients.

24. The computer-implemented data protection method according to claim 12, wherein the explainable AI module includes shapley additive explanations.
